(19) United States
(12) Patent Application Publication
(10) Pub. No.: US 2007/0195968 A1
(43) Pub. Date: Aug. 23, 2007

(54) NOISE SUPPRESSION METHOD AND SYSTEM WITH SINGLE MICROPHONE

(75) Inventor: Marwan Jaber, Montreal, QC (CA)

Correspondence Address: VOLPE AND KOENIG, P.C., UNITED PLAZA, SUITE 1600, 30 SOUTH 17TH STREET, PHILADELPHIA, PA, 19103, US

(73) Assignee: JABER ASSOCIATES, L.L.C., Wilmington, DE (US)

(21) Appl. No.: 11/672,098

(22) Filed: Feb. 7, 2007

Related U.S. Application Data
(60) Provisional application No. 60/771,089, filed on Feb. 7, 2006.

(51) Int. Cl. A61F 11/06; G10K 11/16; H03B 29/00

(52) U.S. Cl. 381/71.11; 381/71.1

(57) ABSTRACT

The present invention is related to a method and apparatus for a robust adaptive algorithm for adjusting coefficients of an adaptive filter and a robust Voice Activity Detector (VAD) which are used in Active Noise Suppressor (ANS). The Filtered Least Mean Squares (LMS) algorithm, which is widely used in digital signal processing, is deployed along with the VAD to reduce the effect of noise in a noisy environment. The present invention guarantees stability of the Filtered LMS algorithm and the VAD, which could be deployed in underground communication system. Numerical simulations and experimentally obtained results exhibit significant improvement on convergence and stability of the proposed adaptive algorithm with application to active noise suppressors.

### CROSS REFERENCE TO RELATED APPLICATION

This application claims the benefit of U.S. Provisional Application No. 60/771,089, filed Feb. 7, 2006 which is incorporated by reference as if fully set forth.

### FIELD OF INVENTION

The present invention is related to a method and apparatus for adjusting coefficients of an adaptive filter. More particularly, the present invention is related to a robustly stabilized algorithm for adaptive filters for use in active noise suppressors.

### BACKGROUND

Different types of adaptive algorithms have been developed and used in conventional adaptive filters such as filtered least mean squares (LMS) algorithms, filtered-x LMS algorithms, filtered normalized least mean squares (NLMS) algorithms and recursive least squares (RLS) algorithms. In particular, the filtered least means square (LMS) algorithm is a popular method for adapting filters due to its simplicity and robustness, and has been adopted in many applications. Adaptive filtering has been applied to such diverse fields as communications, radar, sonar, seismology, and biomedical engineering. In general, adaptive filtering applications typically involve an input vector and a desired response that are used to compute an estimation error, which is then used to control the values of a set of adjustable filter coefficients. The adjustable filter coefficients may take the form of tap weights, reflection coefficients, or rotation parameters, depending on the filter structure employed. As a result of the progress of digital signal processors, it has become practical to implement selective coefficient updates of gradient-based adaptive algorithms.

Although well known and widely used, adaptive filtering applications are not easily understood, and their principles are not easily simplified. Despite the diversity and complexity, adaptive filtering applications, including many practical applications, can be broadly classified. In particular, various applications of adaptive filtering differ in the manner in which the desired response is extracted. In this context, there are four basic classes of adaptive filtering applications, as depicted in FIGS. 1 through 4, and outlined in Table 1.

TABLE 1
Adaptive Filtering Applications

| Adaptive Filtering Class | Application |
|---|---|
| Identification | System Identification |
| | Layered Earth Modeling |
| Inverse Modeling | Predictive Convolution |
| | Adaptive Equalization |
| Prediction | Linear Prediction Coding |
| | Adaptive Differential PCM |
| | Auto Regressive Spectrum Analysis |
| | Signal Detection |
| Interference Canceling | Adaptive Noise Canceling |
| | Echo Cancellation |
| | Radar Polarimetry |
| | Adaptive Beam-forming |

The following notation is used in FIGS. 1-4:
u=input applied to the adaptive filter
y=output of the adaptive filter
d=desired response
e=d−y=estimation error

The functions of the four basic classes of adaptive filtering applications appearing in Table 1 are described further below.

Identification

The notion of a mathematical model is fundamental to sciences and engineering. In the class of applications dealing with identification, an adaptive filter is used to provide a linear model that represents the best fit to an unknown plant as illustrated in FIG. 1. The plant and the adaptive filter are driven by the same input. The plant output supplies the desired responses for the adaptive filter. If the plant is dynamic in nature, the model will be time varying.

Inverse Modeling

In this second class of applications illustrated in FIG. 2, the adaptive filter provides an inverse model representing the best fit to an unknown noisy plant. Ideally, the inverse model has a transfer function equal to the reciprocal of the plant's transfer function. A delayed version of the plant input constitutes the desired response for the adaptive filter. In some applications, the plant input is used without delay as the desired response.

Prediction

In this class of applications illustrated in FIG. 3, the adaptive filter provides the best prediction of the present value of a random signal. The present value of the signal serves the purpose of a desired response for the adaptive filter. Past values of the signal supply the input applied to the adaptive filter. Depending on the application of interest, the adaptive filter output or the estimation error may serve as the system output. In the former case, the system operates as a predictor, and in the latter case, it operates as a prediction error filter.

Interference Cancelling

In this final class of applications, the adaptive filter is used to cancel unknown interference contained in a primary signal, with the cancellation being optimized. The primary signal serves as the desired response for the adaptive filter, and a reference signal is employed as the input to the adaptive filter as illustrated in FIG. 4. The reference signal is derived from a sensor or set of sensors located in relation to the sensor(s) supplying the primary signal in such a way that the information-bearing signal component is weak or essentially undetectable.

Referring more specifically to the application of adaptive noise cancelling, several methods have been proposed in prior art for adaptive noise control employing adaptive filters, where the cancellation of noise is sought by emitting an artificial sound to cancel the unwanted sound at the location of the second measurement device. Theory related to sound propagation and noise cancellation is discussed further below.

When sound waves from a point source strike a plane wall, they produce reflected circular wave fronts as if there were an image of the sound source at the same distance on the other side of the wall. If something obstructs the direct sound from the source from reaching your ear, then it may sound as if the entire sound is coming from the position of the image behind the wall. This kind of sound imaging follows the same laws of reflection as an image in a plane mirror, as illustrated in FIG. 5. The reflection of sound follows the law that states that angle of incidence equals angle of reflection, just like light waves and other waves, and the bounce of a billiard ball off the bank of a table, as in FIG. 6.

The main item of note regarding sound reflections off of hard surfaces is the fact that they undergo a 180-degree phase change upon reflection. This can lead to resonance such as standing waves in rooms. It also implies that the sound intensity near a hard surface is enhanced because the reflected wave adds to the incident wave, giving pressure amplitude that is twice as great in a thin zone near the surface, referred to as the pressure zone. The enhancement of sound intensity in pressure zones is used in pressure zone microphones to increase sensitivity. Referring to FIG. 7, the doubling of pressure gives a 6 decibel increase in the signal picked up by the microphone. Since the reflected wave and the incident wave add to each other while moving in opposite directions, the appearance of propagation is lost and the resulting vibration is called a standing wave. In a similar manner, the modes of vibration associated with resonance in extended objects like strings and air columns have characteristic patterns also called standing waves. These standing wave modes arise from the combination of reflection and interference such that the reflected waves interfere constructively with the incident waves. An important condition for constructive interference is that the waves change phase upon reflection from a fixed end. Under this condition, the medium appears to vibrate in segments or regions and the fact that these vibrations are made up of traveling waves is not apparent, and hence the term standing wave.

Two traveling waves, which exist in the same medium, will interfere with each other as shown in FIG. 8. Referring to FIG. 9, if their amplitudes add, the interference is said to be constructive interference. Otherwise, if they are out of phase and subtract, the interference is referred as destructive interference. Patterns of destructive and constructive interference may lead to dead spots or live spots in auditorium acoustics. Interference of incident and reflected waves is essential to the production of resonant standing waves, such as those shown in FIG. 10.

The sound intensity from a point source of sound will obey the inverse square law if there are no reflections or reverberation, as shown in FIG. 11. Any point source, which spreads its influence equally in all directions without a limit to its range, will obey the inverse square law as a result of geometrical considerations. The intensity of the influence at any given radius r from the source is equal to the source strength divided by the area of the sphere of radius r. Being strictly geometric in its origin, the inverse square law applies to diverse phenomena. For example, point sources of gravitational force, electric field, light, sound or radiation obey the inverse square law. A plot of the intensity drop according to the inverse square law shown in FIG. 12 shows that it drops off rapidly. The plot of FIG. 12 shows the points connected by straight lines but the actual drop is a smooth curve between the points. A plot of the drop of sound intensity according to the inverse square law emphasizes the rapid loss associated with the inverse square law. In an auditorium, such a rapid loss can be unacceptable. However, reverberation in a well-designed auditorium can mitigate it.

Reverberation is the collection of reflected sounds from the surfaces in an enclosure, such as an auditorium as shown in FIG. 13. It is a desirable property of auditoriums to the extent that it helps to overcome the inverse square law drop-off of sound intensity in the enclosure. However, if it is excessive, it can make sounds run together with loss of articulation, such that the sound becomes muddy and garbled.

In prior art (U.S. Pat. No. 6,738,482), in order to cancel unwanted noise, it is necessary to obtain an accurate estimate of the noise to be cancelled. In an open environment where the noise source can be approximated as a point source, background noise can be estimated by microphones spaced as far apart as necessary such that each still receives a substantially similar estimate of the background noise.

In contrast, in a confined environment containing reverberation noise caused by multiple sound reflections, the sound field is very complex and each point in the environment has a very different background noise signal. The further apart the microphones are, the more dissimilar the sound field. As a result, it is difficult to obtain an accurate estimate of the noise to be cancelled in a confined environment by using widely spaced microphones.

If the two microphones are moved closer together, the second microphone should provide a better estimate of the noise to be cancelled in the first microphone. However, if the two microphones are placed very close together, each microphone will cause an additional echo to strike the other microphone. That is, the first microphone will act like a speaker (a sound source) transmitting an echo of the sound field striking the second microphone. Similarly, the second microphone will act like a speaker (a sound source) transmitting an echo of the sound field striking the first microphone. Therefore, the signal from the first microphone, and similarly the second microphone, contain the sum of the background noise plus a reflection of the background noise as illustrated in FIG. 13 and 14, respectively, which results in a poorer estimate of the background noise to be cancelled.

Applicants recognize that there is a need for improved adaptive noise cancellation in confined environments containing reverberation noise caused by sound reflections, where it is difficult to obtain an accurate estimate of background noise.

### SUMMARY

The present invention is a new approach of noise control called active noise suppressor (ANS), which includes a stability-guaranteed algorithm for adaptive filters that can be derived from the strictly positive real property of the error model treated in adaptive system theory. A preferred embodiment of the present invention is a dual microphone noise suppression system in which the echo between two microphones is substantially canceled or suppressed. The assurance of stability of the adaptive system is especially important in the presence of unknown disturbances and mismatch in the order of the adaptive filter. Experimental results, performed on real mining noise, validate the effectiveness of the proposed stable algorithm of the present invention.

### BRIEF DESCRIPTION OF THE DRAWINGS

FIG. 1 is an example of an application of adaptive filters for identification providing a linear model that represents the best fit to an unknown plant.

FIG. 2 is an example of an application of adaptive filters that provides an inverse model representing the best fit to an unknown noisy plant.

FIG. 3 is an example of an application of adaptive filters for providing the best prediction of the present value of a random signal.

FIG. 4 is an example of an application of adaptive filters used to cancel unknown interference contained in a primary signal.

FIG. 5 is an example of a point source of sound reflecting from a plane surface.

FIG. 6 is an example of wave reflection.

FIG. 7 is an example of a standing wave in a pressure zone.

FIG. 8 is an example of sound wave interference.

FIG. 9 illustrates examples of in phase and out of phase interference.

FIG. 10 illustrates the fundamental and second harmonic standing waves for a stretched string.

FIG. 11 illustrates sound intensity from a point source of sound obeying the inverse square law when there are no reflections or reverberation.

FIG. 12 illustrates a plot of the sound intensity dropping off rapidly under the inverse square law.

FIG. 13 is an example of reverberant sound as a collection of all the reflected sounds in an auditorium.

FIG. 14 illustrates a simplified model of a signal from a microphone including background noise and reflection of the background noise in accordance with the present invention.

FIG. 15 illustrates a simplified model of a signal from a microphone including background noise and reflection of the background noise in accordance with the present invention.

FIG. 16 is a pictorial representation of the sound field reaching an ear set in accordance with the present invention.

FIG. 17 is a first embodiment noise suppression system in accordance with a first embodiment of the present invention.

FIG. 18 is a second embodiment of a noise suppression system in accordance with the present invention.

FIG. 19 is an alternate embodiment of a noise suppression communications system in accordance with the present invention.

FIG. 20 is a block circuit diagram of a noise suppressor in accordance with the present invention.

FIG. 21 is a block circuit diagram of first and second adaptation systems including a least mean squares (LMS) filter.

FIG. 22 illustrates noise samples introduced as input to the noise cancellation system of the present invention.

FIG. 23 illustrates the output signal of the noise cancellation system of the present invention.

FIG. 24 is a block circuit diagram of an active noise suppressor in accordance with the present invention.

FIG. 25 block circuit diagram of first adaptation systems including a least mean squares (LMS) filter in accordance with the present invention.

FIG. 26 block circuit diagram of second adaptation systems including a least mean squares (LMS) filter in accordance with the present invention.

FIG. 27 is a block circuit diagram of a decisive switch in accordance with the present invention.

FIG. 28 illustrates noise speech introduced as input to the active noise suppressor of the present invention.

FIG. 29 illustrates the clean speech obtained as output of the active noise suppressor of the present invention.

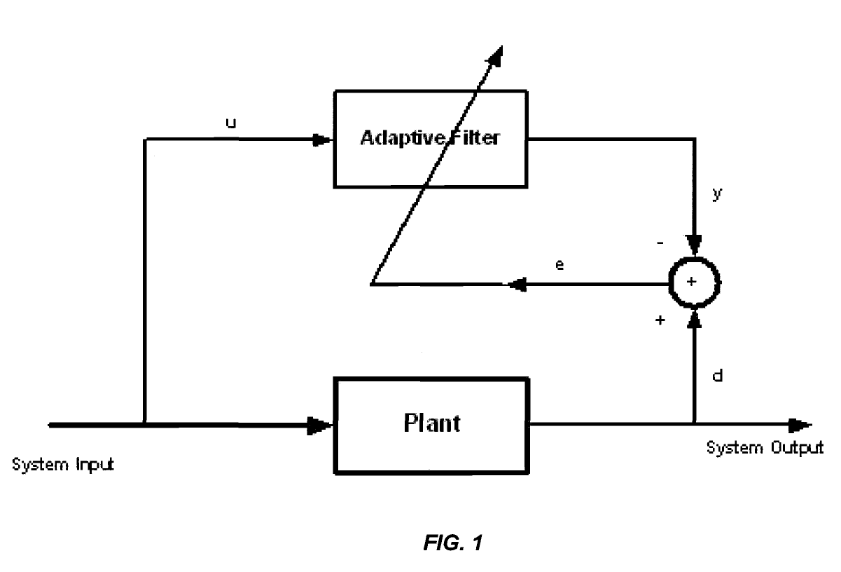

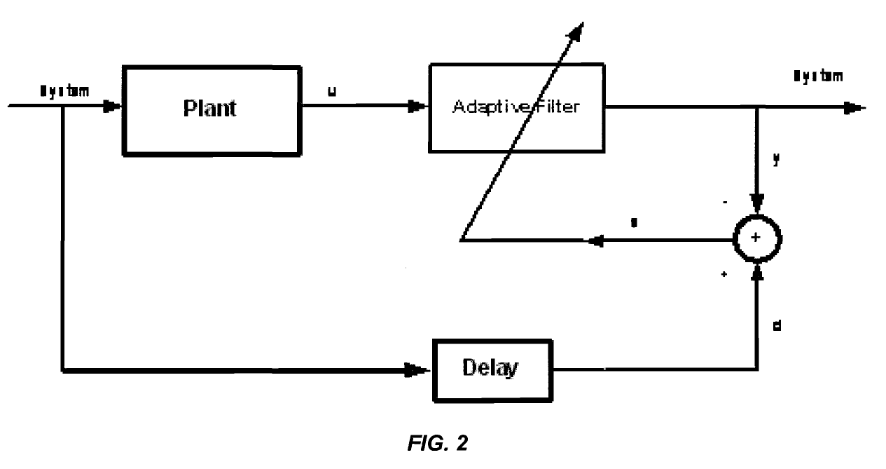

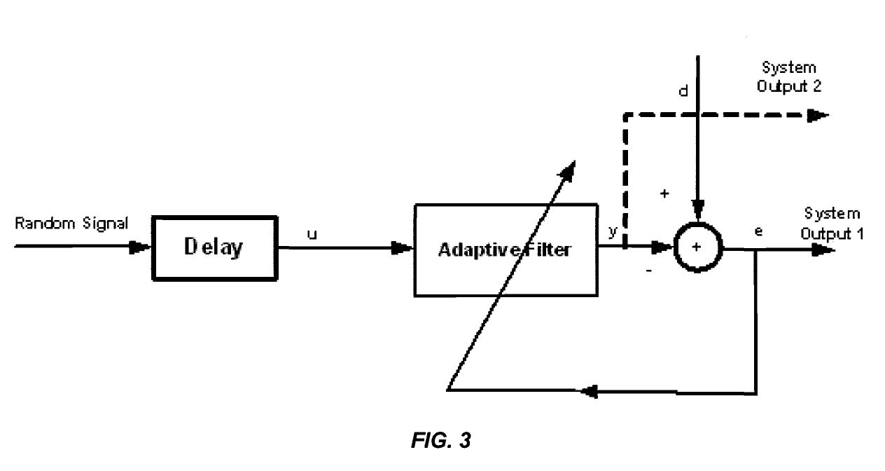

### DETAILED DESCRIPTION OF THE PREFERRED EMBODIMENTS

The present invention proposes a dual microphone noise suppression system in which the echo between the two microphones is substantially canceled or suppressed. Reverberations (i.e. echoes) from one microphone to the other are cancelled by the use of first and second line echo cancellers. Each line echo canceller models the delay and transmission characteristics of the acoustic path between the first and second microphones.

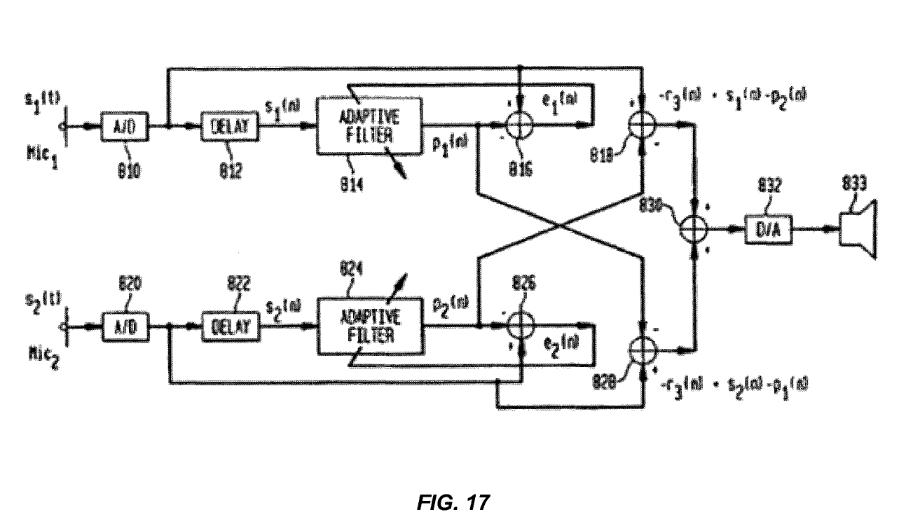

In a preferred embodiment of the present invention, the noise suppression system is part of an ear set to be worn in the outer ear, as shown in FIG. 16. The ear set is a self-contained molded unit, with integral dual microphones, battery, ear canal speaker, signal processing electronics that is convenient to wear and will not interfere with communication and active work. Preferred embodiments are illustrated in FIGS. 17, 18 and 19 and are discussed further below.

In accordance with a preferred embodiment of the present invention, a noise suppression system acts as an ear protector, as shown in FIG. 17, for the purpose of canceling substantially all or most of the noise striking the dual microphones of the ear set.

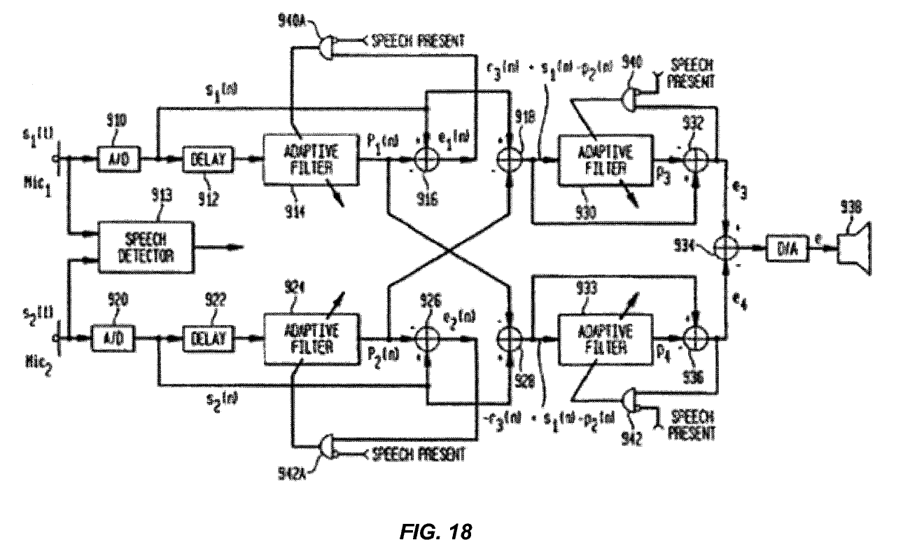

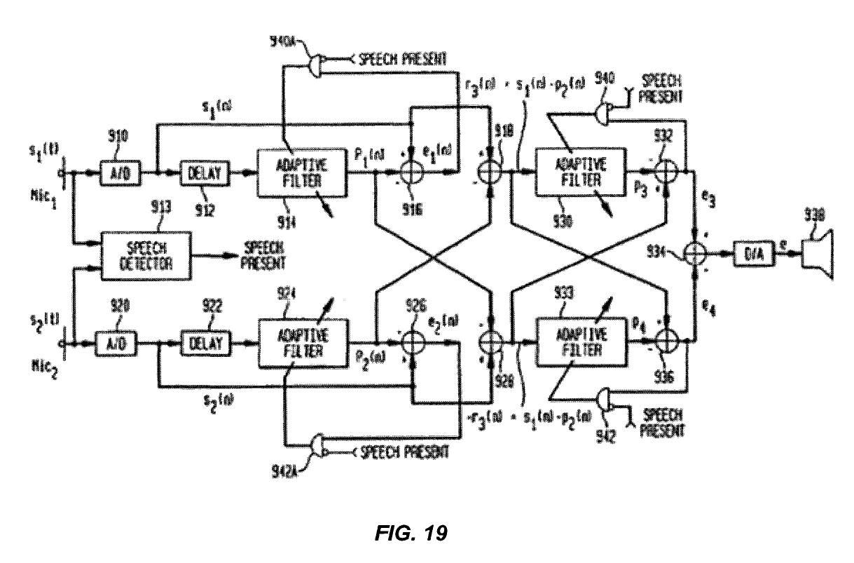

In accordance with another preferred embodiment of the present invention, a noise suppression system acts a noise suppression communication system, suppressing background noise while allowing the desired communication signals to be heard by the wearer. Two possible embodiments are shown in FIGS. 18 and 19.

The conceptual key to the present invention is that the signals received at two closely spaced microphones in a multi-path acoustic environment are each made up of a sum of echoes of the signal received at the other one. This leads to the conclusion that the difference between the two microphone signals is a sum of echoes of the acoustic source in the environment. In the absence of a speech source, the active noise suppressor (ANS) noise control method and system of the present invention first attempts to isolate the difference signal at each of the microphones by subtracting from it an adaptively predicted version of the other microphone signal. It then attempts to adaptively cancel the two difference signals. When speech is present, as detected for example according to a type of voice activity detector (VAD) based strategy, the adaptive cancellation stage has its adaptivity turned off. In other words, the impulse responses of the two FIR filters, one for each microphone, are unchanged for the duration of the speech. The result is that the adaptive canceller does not end up cancelling the speech signal contained in the difference between the two microphone signals.

The crucial task that is facing engineers and scientists is the simplicity of the design, the cost and the size of the products. Therefore, it is a goal of the present invention to reduce the hardware implementation without any losses in the quality of noise cancellation.

In order to cancel unwanted noise, it is necessary to obtain an accurate estimate of the noise to be cancelled. In an open environment, where the noise source can be approximated as a point source, microphones can be spaced far apart as necessary and each will still receive a substantially similar estimate of the background noise. However, in a confined environment containing reverberation noise caused by multiple sound reflections, the sound field is very complex and each point in the environment has a very different background noise signal. The further apart the microphones are, the more dissimilar the sound field. As a result, it is difficult to obtain an accurate estimate of the noise to be cancelled in a confined environment by using widely spaced microphones. The complexity of the problem relies on three factors:
The back ground noise
The complexity of the environment, whether it is an open or closed environment
The deployment of two or more microphones

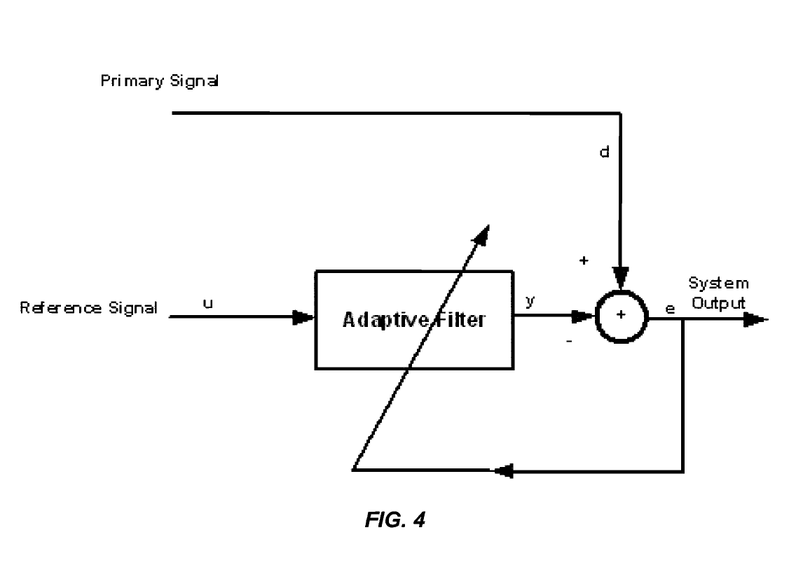

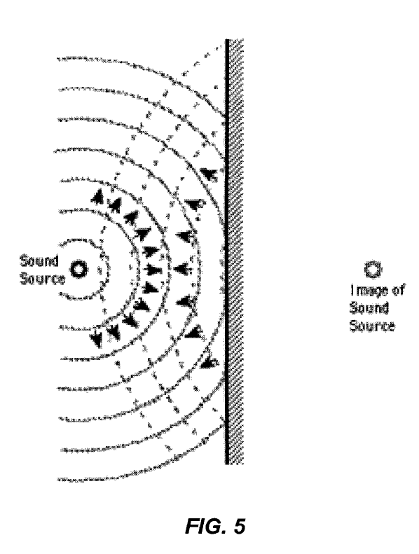

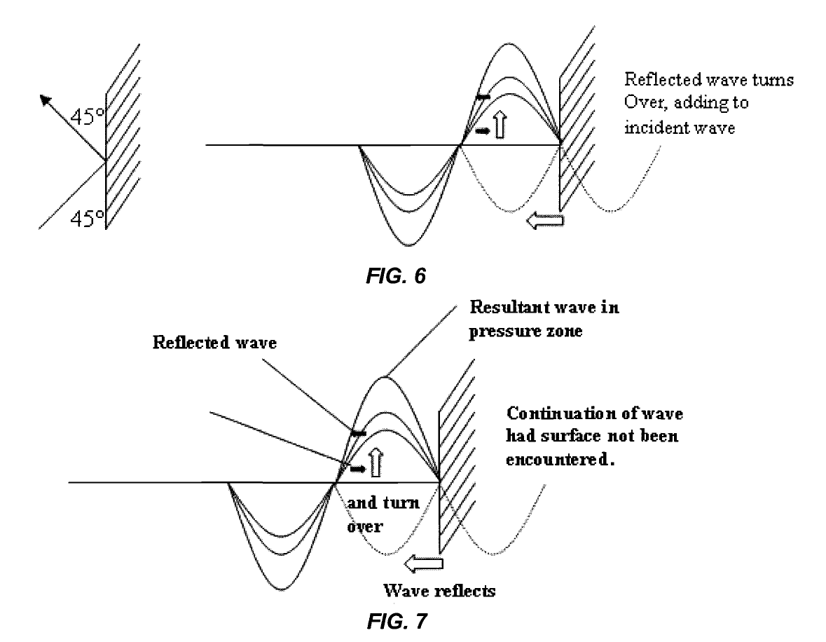

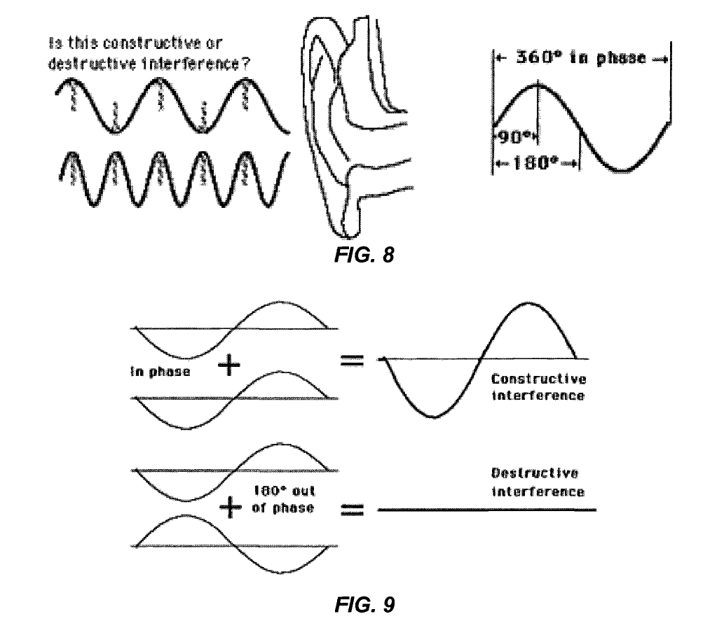

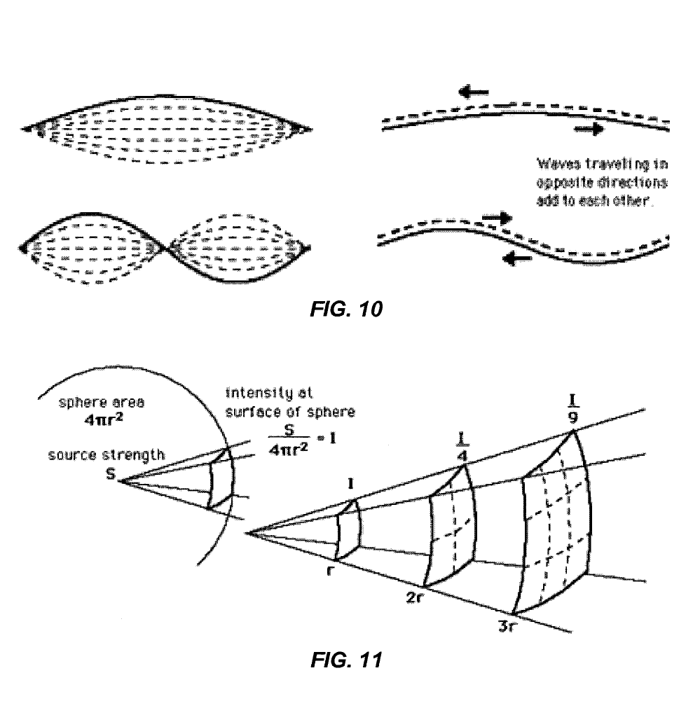

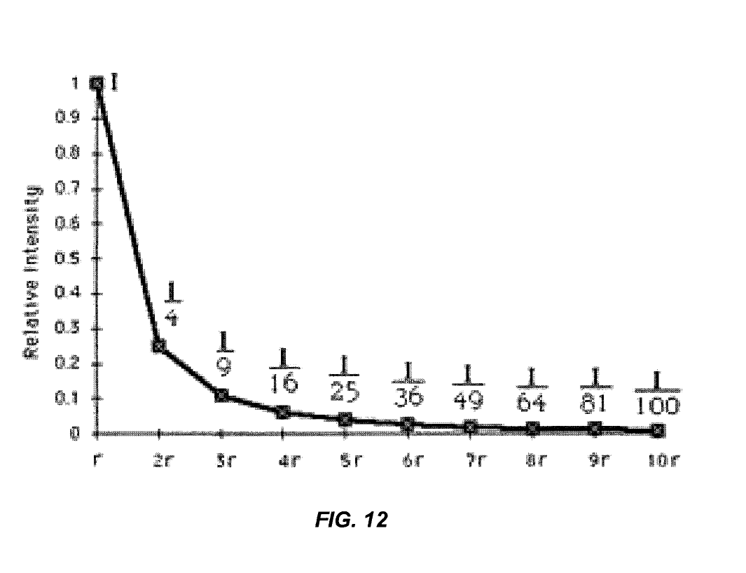

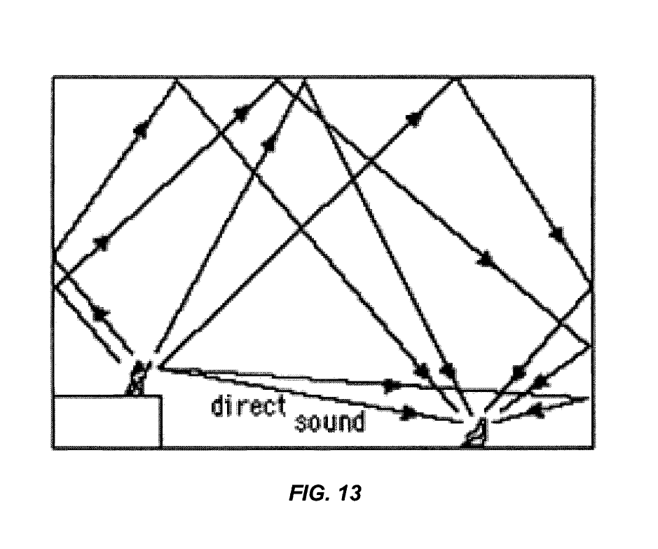

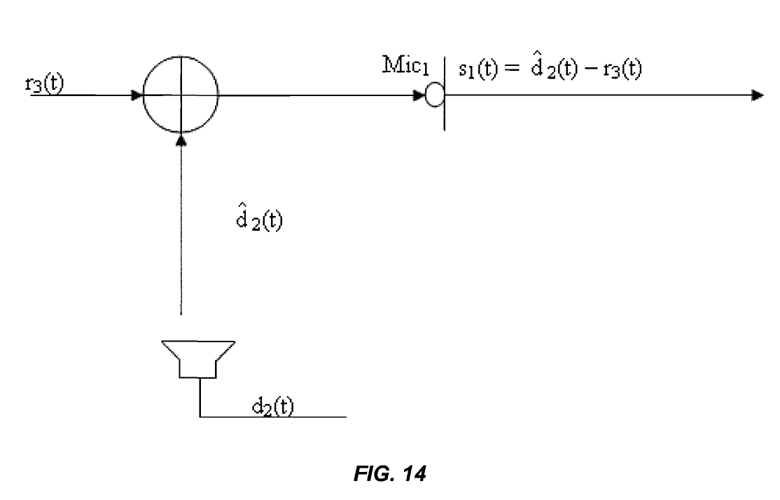

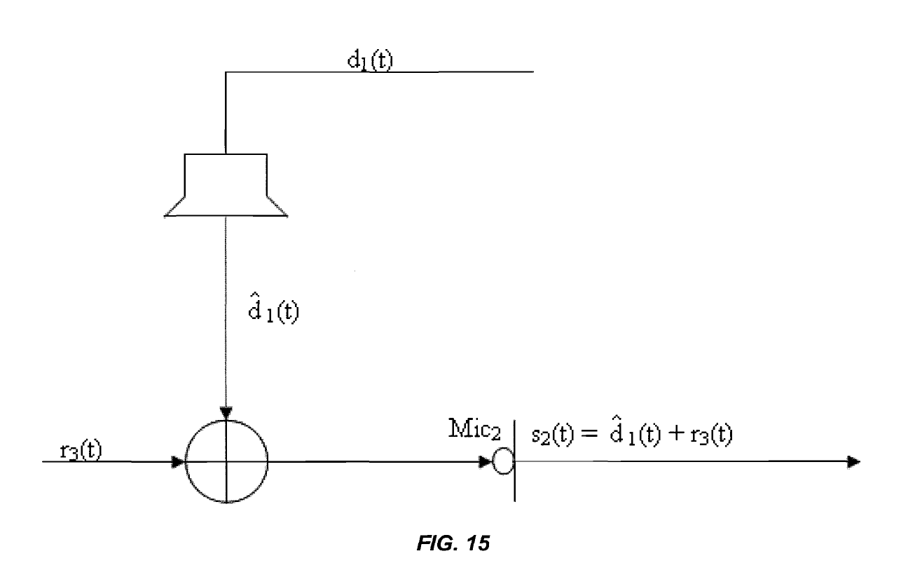

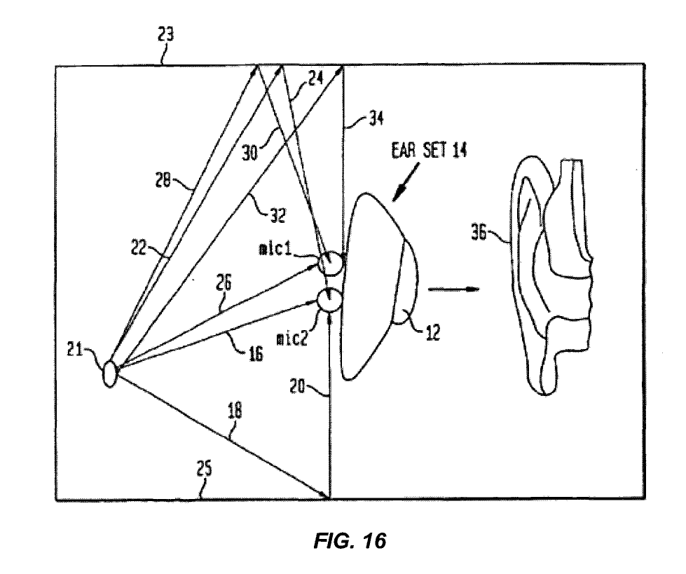

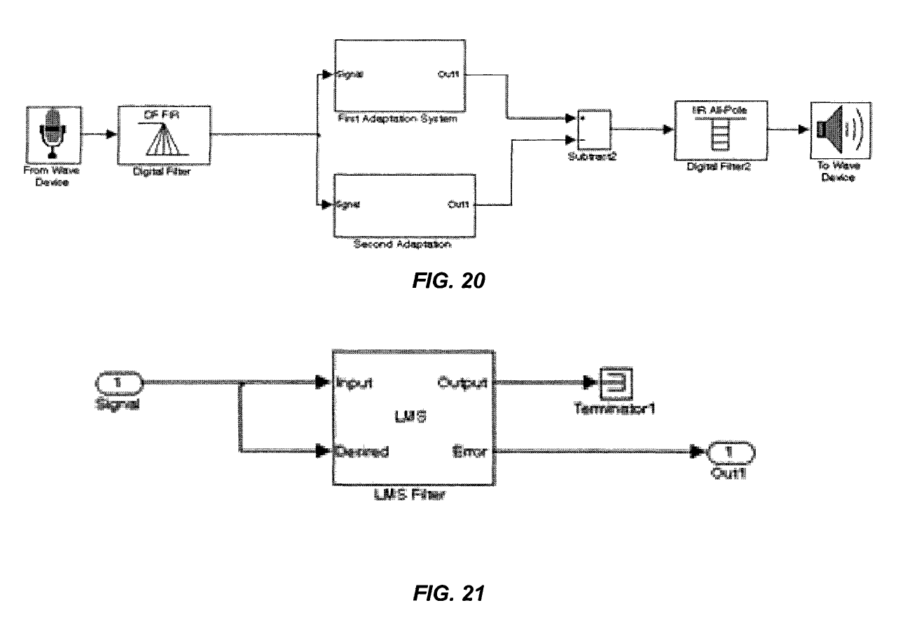

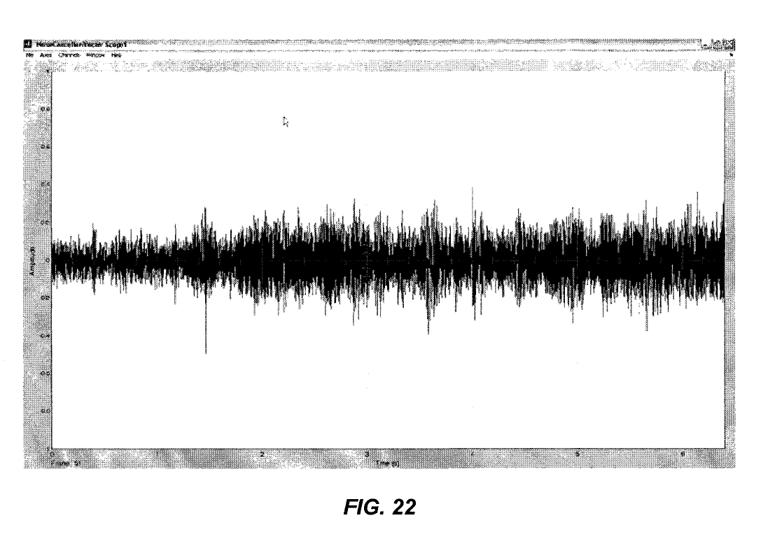

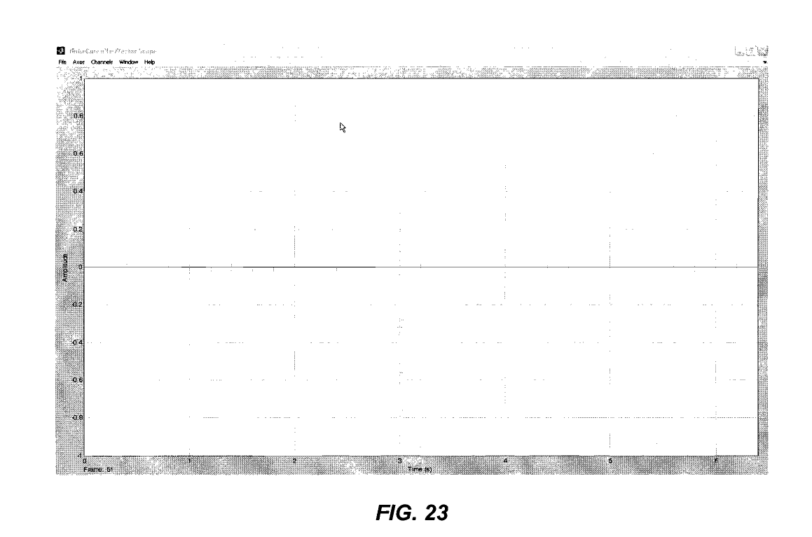

In an open environment, the received signal on the microphone is the direct noise wave, and in a confined environment the received signal on the microphone is the summation of the direct noise signal and the reverberation noise caused by multiple sound reflections. Therefore, by implementing a dual interference canceller, as shown in FIG. 4, on a single microphone as illustrated in FIGS. 20 and 21, a guaranteed stability and conversion to zero is achieved from the first sample as shown by the results in FIGS. 22 and 23.

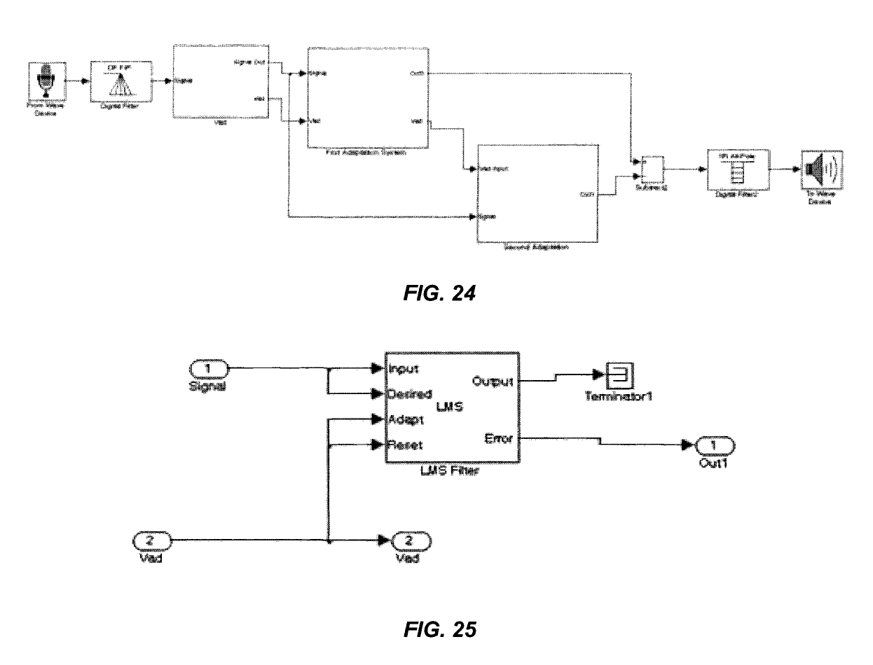

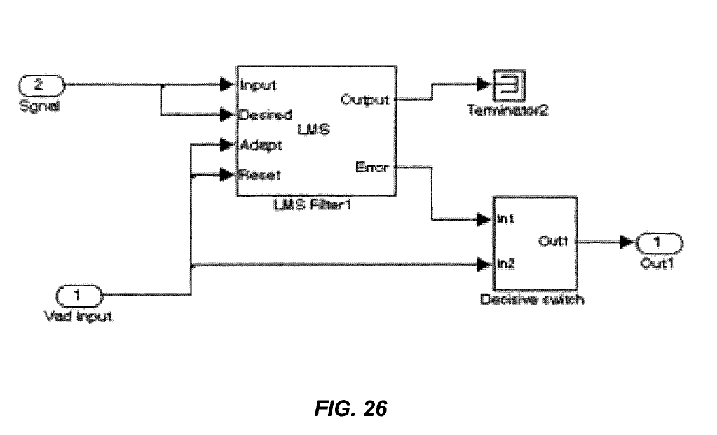

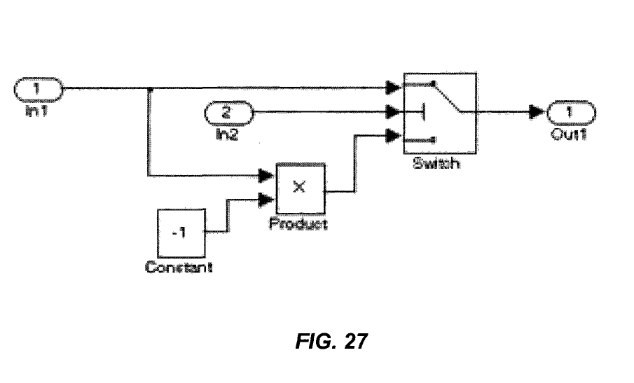

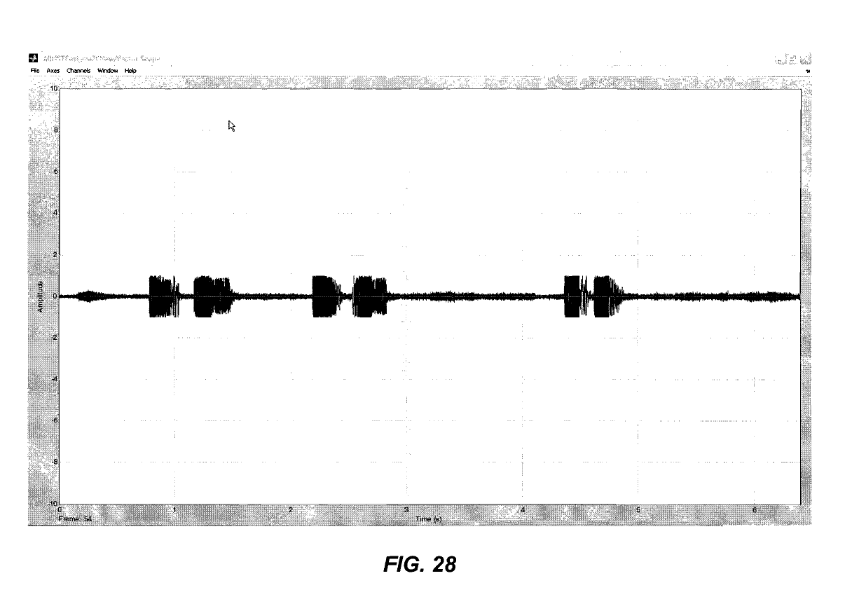

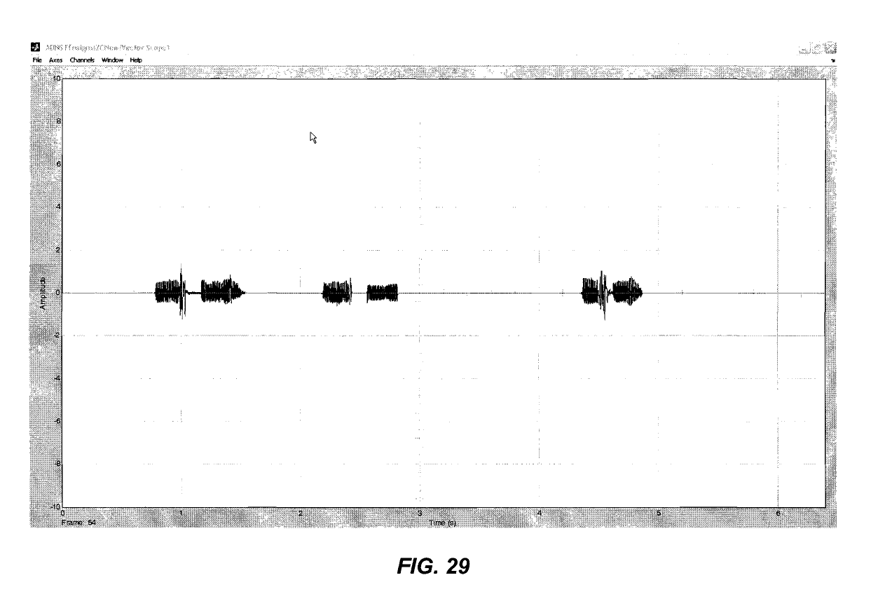

According to the present invention, an active noise suppressor is obtained by using an accurate front-point and end-point detection Voice Activity Detection (VAD) algorithm. By implementing the VAD on the newly proposed noise suppressor with a modification in the second adaptation system, an active noise suppressor is obtained, as illustrated in FIGS. 24, 25, 26 and 27. The simulation results obtained in FIGS. 28 and 29 prove the stability and the convergence of the proposed system by providing clean speech at its output, with the background noise completely eliminated.

Although the features and elements of the present invention are described in the preferred embodiments in particular combinations, each feature or element can be used alone without the other features and elements of the preferred embodiments or in various combinations with or without other features and elements of the present invention.

What is claimed is:

1. An active noise suppressor system for adaptive noise control, the system comprising:
a first microphone configured to receive sound waves including echoes from a second microphone;
the second microphone configured to receive sound waves including echoes from the first microphone;
a first line echo canceller configured to model the delay and transmission characteristics of the received echoes at the first microphone from the second microphone, and to cancel said received echoes; and
a second line echo canceller configured to model the delay and transmission characteristics of the received echoes at the second microphone from the first microphone, and to cancel said received echoes.

2. The system of claim 1 configured to occur in the absence of a speech source.

3. The system of claim 2 wherein:
the first microphone is configured to receive a first signal;
the second microphone is configured to receive a second signal;
the first line echo canceller is configured to isolate a first difference signal by subtracting from the first received signal an adaptively predicted version of an echo signal from the second microphone;
the second line echo canceller is configured to isolate a second difference signal by subtracting from the second received signal an adaptively predicted version of an echo signal from the first microphone;
the system further comprising:
a signal processor configured to cancel the first and second difference signals.

4. The system of claim 1 configured to occur in the presence of a speech source further comprising:
first and second finite impulse response (FIR) filters corresponding to the first and second microphones, respectively, configured to receive and filter speech signals from the respective first and second microphones.

5. The system of claim 4 further comprising:
a voice activity detector (VAD) configured to detect a speech signal according to a voice activity detection algorithm.

6. The system of claim 4 wherein the first and second finite impulse response (FIR) filters are configured to maintain a constant impulse response for the duration of a received speech signal.

7. The system of claim 1 configured as an ear set to be worn in an outer ear.

8. The ear set of claim 7 configured as a self-contained molded unit further comprising:
a battery configured to provide energy to the ear set; and
an ear canal speaker configured to output sound signals into the ear canal.

9. The system of claim 1 configured as a noise suppression communication system wherein the first and second line echo cancellers are configured to cancel respective first and second received background noise signals while preserving desired received communication signals.

10. A method for adaptive noise control and suppression, the method comprising:
receiving a first set of sound waves including echoes from a second set of sound waves;
receiving a second set of sound waves including echoes from the first set of sound waves;
modeling the delay and transmission characteristics of the first set of received sound waves, and canceling said echoes from the second set received sound waves; and
modeling the delay and transmission characteristics of the second set of received sound waves, and canceling said echoes from the first set received sound waves.

11. The method of claim 10 occurring in the absence of a speech source.

12. The method of claim 10 further comprising:
isolating a first difference signal from the first microphone by subtracting from a received signal an adaptively predicted version of an echo signal from the second microphone;
isolating a second difference signal from the second microphone by subtracting from a received signal an adaptively predicted version of an echo signal from the first microphone;
canceling the first and second difference signals by adaptively adjusting filter impulse responses.

13. The method of claim 10 occurring in the presence of a speech source further comprising:
detecting speech signals; and
filtering speech signals from the respective first and second set of received signals.

14. The method of claim 13 wherein the detecting speech signals is according to a voice activity detection algorithm.

15. The method of claim 13 wherein the filtering speech signals is according to a constant impulse response for the duration of a received signal.

16. The method of claim 13 wherein the canceling echoes includes canceling background noise signals while preserving desired received communication signals.
